SEQUENCE LISTING (5 sequences)

SEQ ID NO: 1 - DNA, 100 bp, Artificial Sequence
  Synthetic
  misc_feature (1)..(3): modified with phosphorothioate linker
  misc_feature (1)..(1): may be modified with a 2′O-methyl analog
  misc_feature (98)..(100): modified with phosphorothioate linker
  misc_feature (100)..(100): may be modified with a 2′O-methyl analog
ctgaagttcatctgcaccaccggcaagctgccggtgccctggcccaccctcgtgaccactttgacctatggcgtgcagtgcttcagccgctaccccgacc

SEQ ID NO: 2 - DNA, 100 bp, Artificial Sequence
  Synthetic
  misc_feature (1)..(3): modified with phosphorothioate linker
  misc_feature (1)..(1): may be modified with a 2′O-methyl analog
  misc_feature (98)..(100): modified with phosphorothioate linker
  misc_feature (100)..(100): may be modified with a 2′O-methyl analog
ctgaagttcatctgcaccaccggcaagctgccggtgccctggcccaccctcgtgaccactttgacccatggcgtgcagtgcttcagccgctaccccgacc

SEQ ID NO: 3 - DNA, 100 bp, Artificial Sequence
  Synthetic
  misc_feature (1)..(3): modified with phosphorothioate linker
  misc_feature (1)..(1): may be modified with a 2′O-methyl analog
  misc_feature (98)..(100): modified with phosphorothioate linker
  misc_feature (100)..(100): may be modified with a 2′O-methyl analog
ctgaagttcatctgcaccaccggcaagctgccggtgccctggcccaccctcgtgaccactttgactcatggcgtgcagtgcttcagccgctaccccgacc

SEQ ID NO: 4 - DNA, 100 bp, Artificial Sequence; identical to SEQ ID NO: 2
  Synthetic
  misc_feature (1)..(3): modified with phosphorothioate linker
  misc_feature (1)..(1): may be modified with a 2′O-methyl analog
  misc_feature (64)..(67): modified with phosphorothioate linker
  misc_feature (98)..(100): modified with phosphorothioate linker
  misc_feature (100)..(100): may be modified with a 2′O-methyl analog

SEQ ID NO: 5 - DNA, 100 bp, Artificial Sequence; identical to SEQ ID NO: 1
  Synthetic
  misc_feature (1)..(3): modified with phosphorothioate linker
  misc_feature (1)..(1): may be modified with a 2′O-methyl analog
  misc_feature (64)..(67): modified with phosphorothioate linker
  misc_feature (98)..(100): modified with phosphorothioate linker
  misc_feature (100)..(100): may be modified with a 2′O-methyl analog

(19) United States
(12) Patent Application Publication
(10) Pub. No.: US 2021/0079387 A1
(43) Pub. Date: Mar. 18, 2021

(54) CLEAVAGE-RESISTANT DONOR NUCLEIC ACIDS AND METHODS OF USE

(71) Applicant: The Jackson Laboratory, Bar Harbor, ME (US)

(72) Inventors: William Skarnes, Bar Harbor, ME (US); Justin McDonough, Bar Harbor, ME (US); Enrica Pellegrino, Bar Harbor, ME (US)

(73) Assignee: The Jackson Laboratory, Bar Harbor, ME (US)

(21) Appl. No.: 16/999,649

(22) Filed: Aug. 21, 2020

Related U.S. Application Data
(60) Provisional application No. 62/892,407, filed on Aug. 27, 2019.

(51) Int. Cl. C12N 15/11; C12N 15/90; C12N 9/22; C12N 5/074; C12N 5/073; C12N 5/10

(52) U.S. Cl. CPC C12N 15/11; C12N 15/907; C12N 9/22; C12N 5/0696; C12N 5/0604; C12N 2523/00; C12N 2310/20; C12N 2800/80; C12N 2310/315; C12N 2310/321; C12N 2510/00; C12N 5/10

(57) ABSTRACT

Modeling human disease in cultured cells requires efficient modification of one or both alleles depending on dominant or recessive inheritance. Editing of single nucleotide variants (SNVs) is particularly challenging because the modified allele contains only one mismatch relative to the target sequence and is subject to re-cleavage and damage by the nuclease. The donor nucleic acids provided herein are, inter alia, useful for editing genome sequences by introducing precise changes in a target site in the presence of the donor sequence and minimizing re-cleavage of the donor nucleic acid by nucleases. By doing so, the compositions and methods provided herein produce modified genomic sequences resistant to nuclease cleavage.

### RELATED APPLICATION

This application claims the benefit under 35 U.S.C. § 119(e) of U.S. provisional application No. 62/892,407, filed Aug. 27, 2019, which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Oct. 9, 2020, is named J022770076US01-SEQ-JXV and is 4 kilobytes in size.

### BACKGROUND

The development of programmable nucleases, exemplified by Clustered Regularly Interspace Palindromic Repeats (CRISPR)-Cas9, has revolutionized genetic engineering of virtually any eukaryotic cell or embryo. The introduction of a double strand break (DSB) at a desired genomic site can be repaired via a variety of cellular DNA repair mechanisms. The most frequent outcome of DSB repair is the generation of small insertions/deletions (indels) through error-prone non-homologous end-joining (NHEJ). In the presence of an exogenous DNA repair template containing sequences homologous to the region around the DSB, it is possible to introduce precise genetic changes via homology directed repair (HDR) pathways.

### SUMMARY

The present disclosure provides, in some aspects, cleavage-resistant donor nucleic acids (e.g., DNA repair templates), cells, kits, and methods of use for high-efficiency gene editing, and in some embodiments, high-efficiency control of zygosity.

Modeling human disease in cultured cells, for example, requires efficient modification of one or both alleles depending on dominant or recessive inheritance. Most human diseases are caused by single nucleotide changes. Editing of single nucleotide variants (SNVs) is particularly challenging given the fact that the modified allele contains only one mismatch relative to the target sequence and is subject to re-cleavage and damage by the nuclease. The introduction of additional ‘silent’ or ‘blocking’ mutations (e.g., protospacer adjacent motif (PAM) mutations) is a strategy often used to prevent re-cleavage; however, these additional genetic changes may have unintended consequences on gene expression, for example, by altering normal splicing of mRNA in coding regions or changing chromatin structure in non-coding regulatory sequences. High-efficiency CRISPR Cas9 editing of SNVs in human cells has also been shown to result in significant ‘on target’ damage of the modified SNV allele, which was only partially suppressed under modified conditions (See, e.g., Skarnes W C et al. Methods, 2019; vol. 164-165: pp. 18-28). The technology provided herein, in some embodiments, minimizes or prevents re-cleavage and subsequent damage of a modified target site (e.g., SNV) by the nuclease.

Thus, some aspects of the present disclosure provide donor nucleic acids comprising a (at least one) chemical modification within an internal region that comprises a site-specific nuclease cleavage site.

In some embodiments, a chemical modification is with 1 to 10 nucleotides of the site-specific nuclease cleavage site. For example, a chemical modification may be within 1 to 9, 1 to 8, 1 to 7, 1 to 6, 1 to 5, 1 to 4, 1 to 3, 1 to 2, or within 1 nucleotide of (e.g., adjacent to) the cleavage site. In some embodiments, a chemical modification is within 1 to 5 nucleotides of the site-specific nuclease cleavage site. In some embodiments, a chemical modification is within the site-specific nuclease cleavage site.

In some embodiments, a chemical modification is a phosphorothioate (PS) linkage. Other chemical modifications may be used, many of which are well-known in the art, including, for example, 2′O-methyl analogs and peptide nucleic acids (PNAs). Other chemical modifications are described herein.

In some embodiments, an internal region of the donor nucleic acid comprises 1 to 5 chemical modifications. For example, an internal region of the donor nucleic acid may comprise 1, 2, 3, 4, or 5 chemical modification(s). If a donor nucleic acid includes more than one chemical modification, the chemical modifications may be the same or different relative to one another. For example, an internal region may include two or three PS linkages, or a PS linkage and a 2′O-methyl analog. Other combinations of chemical modifications may be used.

In some embodiments, a donor nucleic acid is single stranded. For example, a donor nucleic acid may be referred to herein as a single-stranded oligonucleotide (ssODN, e.g., having a length of 20 to 200 nucleotides). In other embodiments, a donor nucleic acid is double stranded (has two DNA strands hybridized to each other) or comprises both single-stranded and double-stranded regions.

In some embodiments, a donor nucleic acid comprises a genetic modification relative to a target site. A donor nucleic acid may comprise more than one genetic modification, for example, two, three, four, five, or more genetic modifications. Non-limiting examples of genetic modifications include insertions, deletions, substitutions, and combinations thereof, for example, “indels,” which include an insertion and a deletion of sequence. In some embodiments, a genetic modification is an insertion. In some embodiments, a genetic modification is a deletion. In some embodiments, a genetic modification is a combination of an insertion and a deletion, e.g., an indel. In some embodiments, a genetic modification is a substitution, such as a single nucleotide variant (SNV).

In some embodiments, a site-specific nuclease cleavage site is selected from the group consisting of programmable nuclease cleavage sites and meganuclease cleavage sites. In some embodiments, a site-specific nuclease cleavage site is a meganuclease cleavage site. In some embodiments, a site-specific nuclease cleavage site is a programmable nuclease cleavage site. Non-limiting examples of programmable nuclease cleavage sites include Cas9 nuclease cleavage sites, zinc finger nuclease (ZFN) cleavage sites, and transcription activator-like effector nuclease (TALEN) cleavage sites. In some embodiments, a programmable site-specific nuclease cleavage site is a Cas9 nuclease cleavage site. In some embodiments, a programmable site-specific nuclease cleavage site is a ZFN cleavage site. In some embodiments, a programmable site-specific nuclease cleavage site is a TALEN cleavage site.

In some embodiments, a donor nucleic acid comprises a chemical modification within an end region of the donor nucleic acid (e.g., within 1 to 5 nucleotides of the terminal 5′ phosphate or the terminal 3′ hydroxyl group). In some embodiments, a donor nucleic acid comprises a chemical modification within a 5′ end region. For example, a donor nucleic acid may include a chemical modification at the 5′ terminal nucleotide or within 5 nucleotides of the 5′ terminal nucleotide. In some embodiments, a donor nucleic acid comprises a chemical modification within a 3′ end region. For example, a donor nucleic acid may include a chemical modification at the 3′ terminal nucleotide or within 5 nucleotides of the 3′ terminal nucleotide. Any of the chemical modifications described or contemplated herein may be used. In some embodiments, a chemical modification within an end region is a (at least one) PS linkage. In some embodiments, a chemical modification within an end region is a (at least one) 2′O-methyl analog.

Other aspects provide kits that comprise a donor nucleic acid comprising a chemical modification within an internal region that comprises a site-specific nuclease cleavage site. A kit may further comprise a site-specific nuclease or a nucleic acid (e.g., DNA) encoding a site-specific nuclease. In some embodiments, a kit comprises a programmable nuclease (e.g., Cas9 nuclease, ZFN, and/or TALEN). In some embodiments, a kit comprises a meganuclease. In some embodiments, a kit comprises a guide RNA (gRNA) or a nucleic acid (e.g., DNA) encoding a gRNA.

Yet other aspects provide methods comprising delivering to a cell that comprises a target site a donor nucleic acid comprising a chemical modification within an internal region that comprises a site-specific nuclease cleavage site, wherein the cell comprises a target site (e.g., a genomic target sequence). In some embodiments, a donor nucleic acid comprises a sequence that is homologous (at least partially homologous) to the target site. In some embodiments, the cell also comprises a site-specific nuclease (e.g., programmable nuclease and/or meganuclease) that can cleave the target site. In some embodiments, the methods further comprise delivering to the cell a site-specific nuclease or a nucleic acid (e.g., DNA) encoding a site-specific nuclease that can cleave the target site. In some embodiments, the methods further comprise delivering to the cell a gRNA that can bind to the target site or a nucleic acid (e.g., DNA) encoding a gRNA that can bind to the target site.

Yet other aspects provide methods, for example, for controlling zygosity in a cell (e.g., mammalian cell, such as human cell or rodent cell, e.g., mouse cell), comprising delivering to a cell that comprises a target site within a target allele a mixture of donor nucleic acids of the present disclosure. In some embodiments, a subset of donor nucleic acids of the mixture comprise a wild-type allele and a subset of donor nucleic acids of the mixture comprise a single nucleotide variant allele, relative to the target allele. In some embodiments, a subset of donor nucleic acids of the mixture comprise a first single nucleotide variant allele and a subset of donor nucleic acids of the mixture comprise a second single nucleotide variant allele, relative to the target allele.

In some embodiments, the methods further comprise maintaining the cell under conditions that result in cleavage of the target site (e.g., under suitable culture conditions for cell survival and nuclease activity). In some embodiments, the methods further comprise maintaining the cell under conditions that result in the target site comprising the chemical modification. In some embodiments, the conditions include a cold shock. In some embodiments, the conditions include the presence of a small molecule enhancer of homology directed repair (HDR). See, e.g., Skarnes W C et al. Methods, 2019; vol. 164-165: pp. 18-28.

Still other aspects provide cells comprising a target site and a donor nucleic acid comprising a chemical modification within an internal region that comprises a site-specific nuclease cleavage site. In some embodiments, a donor nucleic acid comprises sequence that is homologous to (at least partially homologous to, e.g., includes a genetic modification relative to) the target site. In some embodiments, a cell further comprises a site-specific nuclease or a nucleic acid (e.g., DNA) encoding a site-specific nuclease that can cleave the target site. In some embodiments, a cell further comprises a gRNA or a nucleic acid (e.g., DNA) encoding a gRNA that can bind to the target site.

In some embodiments, a cell is a human cell. In some embodiments, a cell is a rodent cell. In some embodiments, a cell is a stem cell (e.g., embryonic stem cell). In some embodiments, a cell is a pluripotent stem cell. In some embodiments, a cell is an induced pluripotent stem cell (iPSC). In some embodiments, a cell is a human iPSC.

Further aspects provide zygotes comprising a target site and a donor nucleic acid comprising a chemical modification within an internal region that comprises a site-specific nuclease cleavage site. In some embodiments, a zygote further comprises site-specific nuclease or a nucleic acid encoding a site-specific nuclease that can cleave the target site. In some embodiments, a zygote further comprises a guide RNA (gRNA) or a nucleic acid encoding a gRNA that can bind to the target site. In some embodiments, a zygote is a human zygote or a rodent zygote. In some embodiments, a zygote is selected from 1-cell stage zygotes, 2-cell stage zygotes, 4-cell stage zygotes, and 8-cell stage zygotes.

### BRIEF DESCRIPTION OF THE DRAWINGS

FIGS. 1A-1C. The figures show homology directed repair (HDR) in a blue fluorescent protein (BFP) to green fluorescent protein (GFP) assay using unmodified and modified single-stranded oligonucleotide (ssODN) repair templates. Human induced pluripotent stem (iPS) cells were nucleofected with high-fidelity Cas9 RNP targeting BFP sequence and end-modified ssODN encoding a single nucleotide variant converting BFP to GFP (H67Y) and wild-type (WT) BFP sequence (FIG. 1A), the H67Y variant and wild-type BFP sequence containing a silent coding variant (FIG. 1B), and cleavage-resistant ssODN forms of H67Y and WT ssODN (Cr—H67Y and Cr-WT) containing three internal phosphorothioate linkages at the predicted Cas9 cut site (FIG. 1C). The ssODN sequences used are shown in FIG. 2.

FIG. 2. The figure shows the sequences of end-modified and cleavage-resistant ssODN templates. Single nucleotide variants of BFP are boxed, phosphorothioate linkages are indicated with an asterisk, the PAM sequence is underlined, and the arrow shows the predicted re-cleavage site for Cas9 RNP targeting wild-type BFP sequence. Sequences from top to bottom are SEQ ID NOs: 1-5.

FIG. 3. This figure shows the results of a BFP to GFP assay using H67Y (C>T) and wildtype ssODN templates with and without phosphorothioate linkages. The cleavage-resistant ssODNs (Cr—H67Y and Cr-WT) contain 3 PS linkages at the predicted Cas9 cleavage site as shown in FIG. 2.

### DETAILED DESCRIPTION

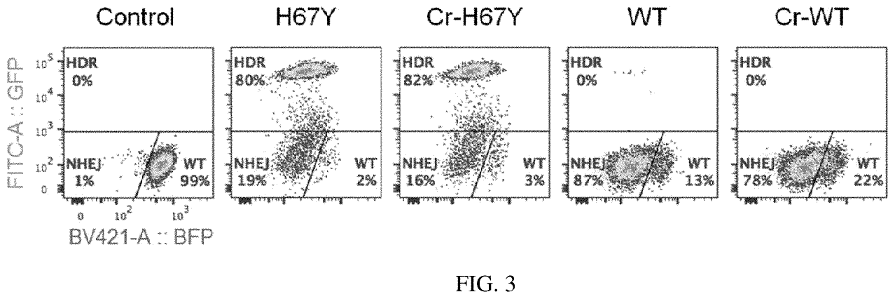

The present disclosure provides, in some aspects, methods and compositions relating to chemically-modified donor nucleic acids (e.g., DNA repair templates) for high-efficiency genomic editing and, in some embodiments, control of zygosity (e.g., recovery of cells of all possible genotypes). As most diseases and risk factors in humans are caused by single nucleotide variants (SNVs), genome editing with programmable nucleases (e.g., CRISPR-Cas9) and other site-specific nucleases has been used to introduce SNVs into cells and embryos. The generation of SNVs by gene editing is challenging because the single nucleotide change is susceptible to re-cleavage by the programmable nuclease and damage by non-homologous end-joining (NHEJ). While the addition of “blocking” or “silent” mutations may be used to prevent re-cleavage, these additional genetic changes may have unintended consequences on gene expression and confound the analysis of the disease model. The donor nucleic acids described herein have been shown to prevent re-cleavage of the edited SNV and improve the overall efficiency of editing (FIG. 3). Cleavage-resistant donor nucleic acids of the present disclosure, in some embodiments, also permit mixing of wildtype (WT) and SNV templates, resulting in more controlled zygosity and more efficient recovery of cells of all possible genotypes (i.e., SNV/SNV, SNV/WT, and WT/WT), obviating the need for blocking mutations.

### Donor Nucleic Acids

A donor nucleic acid is a nucleic acid (e.g., DNA or RNA) that includes a nucleotide sequence of interest (e.g., a modification relative to a target site in a genome) and can be used to modify a target site (e.g., a genomic sequence). Thus, a donor nucleic acid is typically used for gene editing. Examples of donor nucleic acids include DNA repair templates, such as homology-directed repair (HDR) templates and homologous recombination (HR) templates. Methods for designing DNA repair templates are described in the art. See, e.g., Chen F et al. Nat Methods 2011; vol. 8; pp. 753-755; and Mansour S L et al. Nature 1988; vol. 336: pp. 348-352.

In some embodiments, a donor nucleic acid is single stranded (e.g., a single-stranded oligonucleotide (ssODN)). In other embodiments, a donor nucleic acid is double stranded (e.g., a double-stranded plasmid). In yet other embodiments, a donor nucleic acid includes single-stranded sequences and double-stranded sequences.

In some embodiments, a donor nucleic acid comprises a sequence that is partially homologous to a target nucleic acid sequence (in a target site). For example, a donor nucleic acid may comprise a sequence that is 50% to 99% homologous to (e.g., shares 50% to 99% identity with) a target nucleic acid sequence. In some embodiments, a donor nucleic acid comprises a sequence that is at least 50%, 55%, 60%, 65%, 70%, 75%, 80%, 85%, 90%, 91%, 92%, 93%, 94%, 95%, 96%, 97%, 98%, or 99% homologous to a target nucleic acid sequence. A donor nucleic acid, in some embodiments, includes (a) a nucleotide sequence that is non-homologous to a sequence in a target site and (b) flanking nucleotide sequences that are homologous to the target site. For example, example a donor nucleic acid may include a non-homologous sequence flanked by homology arms (e.g., a left homology arm and a right homology arm). Homology arms need not be the same length and may be at least 5 nucleotides, at least 10 nucleotides (e.g., 10 to 200 nucleotides), at least 15 nucleotides (e.g., 15 to 200 nucleotides), at least 20 nucleotides (e.g., 20 to 200 nucleotides), at least 25 nucleotides (e.g., 25 to 200 nucleotides), at least 30 nucleotides (e.g., 30 to 200 nucleotides), at least 35 nucleotides (e.g., 35 to 200 nucleotides), at least 40 nucleotides (e.g., 40 to 200 nucleotides), at least 45 nucleotides (e.g., 45 to 200 nucleotides), or at least 50 nucleotides (e.g., 50 to 200 nucleotides).

In some embodiments, a donor nucleic acid comprises a genetic modification relative to a target site (e.g., to a target nucleic acid sequence within a target site). Various types of genetic modifications are known in the art. Non-limiting examples of genetic modifications that may be present in a donor nucleic acid include insertions, deletions, substitutions, and combinations thereof (e.g., indels), relative to a target site. Insertions into donor nucleic acid sequences may comprise coding regions that produce proteins (e.g., detectable molecules) or non-coding regions that regulate gene expression (e.g., activate gene expression or repress gene expression). Deletions from donor nucleic acid sequences may comprise deleting a portion of the donor nucleic acid sequence relative to the target site. Substitutions in donor nucleic acids include one or more nucleotide change(s). For example, a donor nucleic acid may include at least 1, at least 2, at least 3, at least 4, or at least 5 nucleotide substitutions, relative to a target site. In some embodiments, a substitution is a single nucleotide variant (SNV), relative to a target site (e.g., a wild-type allele sequence).

The length of a donor nucleic acid (single-stranded or double-stranded) may vary. In some embodiments, a donor nucleic acid has a length of 10 to 2,500, 10 to 2000, 10 to 1500, 10 to 1000, 10 to 500, 10 to 250, 10 to 100, or 10 to 50 nucleotides. In some embodiments, a donor nucleic acid has a length of 20 to 2,500, 20 to 2000, 20 to 1500, 20 to 1000, 20 to 500, 20 to 250, 20 to 100, or 20 to 50 nucleotides. In some embodiments, a donor nucleic acid has a length of 10, 15, 20, or 25 nucleotides. In some embodiments, a donor nucleic acid has a length of 50 nucleotides. In some embodiments, a donor nucleic acid has a length of 100 nucleotides. In some embodiments, a donor nucleic acid has a length of 150 nucleotides. In some embodiments, a donor nucleic acid has a length of 200 nucleotides. In some embodiments, a donor nucleic acid has a length of 500 nucleotides. In some embodiments, a donor nucleic acid has a length of 1,000 nucleotides. In some embodiments, a donor nucleic acid has a length of 2,000 nucleotides.

Nucleic Acid Regions—Internal and End Regions

A donor nucleic acid of the present disclosure comprises an internal region that comprise a site-specific nuclease cleavage site. Herein, a donor nucleic acid, such as a linear donor nucleic acid, for example, includes an internal region flanked by a 5′ (upstream) end region (with a terminal phosphate) and a 3′ (downstream) end region (with a terminal hydroxyl group). A 5′ end region, in some embodiments, includes 1 to 5 nucleotides (e.g., 1, 2, 3, 4, or 5 nucleotides) at the most 5′ end of a donor nucleic acid. A 3′ end region, in some embodiments, includes 1 to 5 nucleotides (e.g., 1, 2, 3, 4, or 5 nucleotides) at the most 3′ end of a donor nucleic acid. Thus, an internal region is the remaining intervening sequence of the donor nucleic acid, located between (and in some embodiments contiguous with) the 5′ end region and the 3′ end region. The length of an internal region can vary and depends on the length of the donor nucleic acid. In some embodiments, an internal region has a length of 5 to 2,500, 5 to 2000, 5 to 1500, 5 to 1000, 5 to 500, 5 to 250, 5 to 100, or 5 to 50 nucleotides.

Site-Specific Nuclease Cleavage Sites

An internal region of a donor nucleic acid comprise a (at least one) site-specific nuclease cleavage site. A nuclease cleavage site is the location at which a strand of DNA is cleaved (broken) by a nuclease. A site-specific nuclease cleavage site comprises a nucleotide sequence that can be specifically recognized and cleaved by a site-specific nuclease, e.g., hydrolytically cleaved at a specific glycosidic bond or a sugar-phosphate ester bond of the nucleic acid. The cleavage may be single-stranded or double-stranded, depending on the particular site-specific nuclease used. Consensus sequences for site-specific nuclease cleavage sites (e.g., meganucleases and/or programmable nucleases) are known, any of which may be used in accordance with the present disclosure. In some embodiments, a site-specific nuclease cleavage site comprises a sequence that is homologous to (e.g., identical to) a consensus sequence of a site-specific nuclease cleavage site. In other embodiments, a site-specific nuclease cleavage site shares 50% to 100% identity (e.g., 60% to 100%, 70%-100%, 80% to 100%, or 90% to 100% identity) with a consensus sequence of a site-specific nuclease cleavage site. In some embodiments, a site-specific nuclease cleavage site shares 99%, 98%, 97%, 96%, 95%, 94%, 93%, 92%, 91%, 90%, 85%, 80%, 75%, 70%, 65%, 60%, 55%, or 50% identity with a consensus sequence of a site-specific nuclease cleavage site. Site-specific nucleases and their consensus cleavage sites are described elsewhere herein. It should be understood that an internal region of a donor nucleic acid may comprise more than one site-specific nuclease cleavage site. For example, a donor nucleic acid may comprise two or three site-specific nuclease cleavage sites.

Chemical Modifications

A donor nucleic acid of the present disclosure comprises a (at least one) chemical modification within an internal region that comprise a site-specific nuclease cleavage site. A chemical modification as used herein renders the donor nucleic acid resistant to (e.g., minimizes/reduces the frequency of or prevents) site-specific nuclease activity (e.g., exonuclease or endonuclease activity). In some embodiments, a chemical modification, while present at or near a site-specific nuclease cleavage site, does not alter the nucleotide sequence of the cleavage site. In some embodiments, a chemical modification within an internal region is located 1 to 10 nucleotides, or 1 to 5 nucleotides, upstream from and/or downstream from a site-specific nuclease cleavage site. For example, a chemical modification within an internal region may be located 1, 2, 3, 4, 5, 6, 7, 8, 9, or 10 nucleotides upstream from or downstream from a site-specific nuclease cleavage site.

In some embodiments, an internal region of a donor nucleic acid comprises a (at least one) PS linkage at or near a nuclease cleavage site. In some embodiments, an internal region of a donor nucleic acid comprises 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, or 9 to 10 chemical modifications (e.g., PS linkages). In some embodiments, an internal region of a donor nucleic acid comprises 1, 2, 3, 4, 5, 6, 7, 8, 9, 10, 11, 12, 13, 14, 15, 16, 17, 18, 19, 20, or more chemical modifications (e.g., PS linkages). In some embodiments, an internal region of a donor nucleic acid comprises 1 to 5 internal PS linkages. In some embodiments, an internal region of a donor nucleic acid comprises three PS linkages.

In some embodiments, a donor nucleic acid also comprises a chemical modification in an end region, for example, in a 5′ end region or in a 3′ end region. In some embodiments, a donor nucleic acid comprises a chemical modification in a 5′ end region and a chemical modification in a 3′ end region. In some embodiments, an end region of a donor nucleic acid comprises 1 to 5 chemical modifications (e.g., PS linkages). In some embodiments, the 5′ end region and/or the 3′ end region of a donor nucleic acid comprises two terminal PS linkages—for example, linking together the 5′ terminal three nucleotides and/or linking together the 3′ terminal three nucleotides).

Chemical modifications may include any change to a nucleic that confers resistance to (e.g., minimizes/reduces the frequency of or prevents) cleavage of the nucleic acid by a site-specific nuclease. In some embodiments, chemical modifications include nucleotide analogs or modified backbone residues or linkages. Chemical modifications may be naturally occurring or non-naturally occurring (e.g., synthetic). In some embodiments, a chemical modification comprises a modified linkage. Examples of modified linkages include, but are not limited to, phosphodiester derivatives including, e.g., phosphoramidate, phosphorodiamidate, phosphorothioate (also known as phosphothioate, PS), phosphorodithioate, phosphonocarboxylic acids, phosphonocarboxylates, phosphonoacetic acid, phosphonoformic acid, methyl phosphonate, boron phosphonate, and O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)). In some embodiments, the internucleotide linkages in DNA are phosphorothiate (PS), phosphodiester, or a combination of both. In some embodiments, internal chemical modifications include PS linkages.

Other examples of chemical modifications include 2′-O-modifications, such as pentyl, propyl, methyl, and fluoro modifications. In some embodiments, internal chemical modifications (located within the internal region) include 2′-O-methyl analogs. In some embodiments, terminal chemical modifications (located in an end region) include 2′-O-methyl analogs. In some embodiments, chemical modifications include modified nucleotides such as 2-aminopurine, 2,6-diaminopurine, inverted dT, inverted dideoxy-T, dideoxy-C, 5-methyl dC, deoxylnosine, 5-hydroxybutynl-2′-deoxyuridine, 8-aza-7-deazaguanosine, 5-nitroindole, hydroxymethyl dC, iso-dC, iso-dG, 2-methoxyethoxy A, 2-methoxyethoxy MeC, 2-methoxyethoxy G, 2-methoxyethoxy T. These modified nucleotides are all known in the art and publically available, for example, through Integrated DNA Technologies (idtdna.com/pages/products/custom-dna-rna/oligo-modifications).

The donor nucleic acid may be or include, for example deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof. In some embodiments, the donor nucleic acid is a DNA.

### Site-Specific Nucleases

The site-specific nuclease cleavage sites described herein are cleaved by cognate site-specific nucleases. A nuclease, generally, is an enzyme that cleaves a nucleic acid into smaller units. Without wishing to be bound by theory, it is thought that a chemical modification at (or near) a site-specific nuclease cleavage site of a donor nucleic acid renders the nucleic acid resistant to site-specific nuclease activity (e.g., exonuclease or endonuclease activity). A nucleic acid is considered to be resistant to cleavage by a nuclease if the nucleic acid cannot be cleaved by the nuclease, or the frequency at which the nucleic acid is cleaved by the nuclease is reduced, for example, by least 50% (e.g., at least 55%, at least 60%, at least 65%, at least 70%, at least 75%, at least 80%, at least 85%, at least 90%, or at least 95%). Therefore, in some embodiments, a site-specific nuclease is used to cleave a target site, for example, in genomic DNA (e.g., of a host cell), but does not cleave the corresponding chemically-modified target site in the internal region of the donor nucleic acid. Non-limiting examples of site-specific nucleases that may be used as provided herein include meganucleases and programmable nucleases.

Meganucleases

Meganucleases, also referred to as homing endonucleases, recognize a double-stranded DNA sequence of 12 to 40 base pairs. There are five families of meganucleases: LAGLIDADG, GIY-YIG, HNH, His-Cys box, and PD-(D/E)XK. The families are delineated by sequence and structure motifs. Non-limiting examples of meganucleases include I-Sce I, I-Ceu I, I-Chu I, I-Cre I, i-Csm I, I-Dir I, I-Dmo I, I-Hmu I, I-Hmu II, I-Ppo I, I-Sce II, I-Sce III, I-Sce IV, I-Tev I, I-Tev II, I-Tev III, PI-Mle I, PI-Mtu I, PI-Pfu I, PI-Psp I, PI-Tli I, PI-Tli II, and PI-Sce V. Other meganucleases are known in the art and may be accessed, for example from databases such as homingendonuclease.net (Taylor et al., Nucleic Acids Res. 40(W1):W110-W116). Engineered meganucleases are also contemplated herein. See, e.g., Silva et al. Curr Gene Ther. 2011 February; 11(1): 11-27, incorporated herein by reference.

Programmable Nucleases

Programmable nucleases (also known as targeted nucleases; see, e.g., Porter et al. Compr Physiol. 2019 Mar. 14; 9(2):665-714); Kim et al. Nat Rev Genet. 2014 May; 15(5):321-34; and Gaj et al. Trends Biotechnol. 2013 July; 31(7):397-405) include, for example, zinc finger nucleases (ZFNs), transcription activator-like effector nucleases (TALENs), and RNA-guided nucleases, such as Cas9 and Cpf1 nucleases. It should be understood that the aspects and embodiments provided herein that encompass “nucleases” also encompass “nickases.” A nickase is a type of nuclease. Thus, a Cas9 nickase is a type of Cas9 nuclease. In some embodiments, a programmable nuclease is a ZFN. In some embodiments, a programmable nuclease is a TALEN. In some embodiments, a programmable nuclease is a Cas9 nuclease (e.g., that introduces a double-strand break in DNA, i.e., cleaves the sense strand and the antisense strand). For example, the Cas9 nuclease may be a Cas9 nickase (introduces a single-strand break in DNA, i.e., cleaves the sense strand or the antisense strand).

In some embodiments, programmable nucleases are guided to a target sequence by protein DNA binding domains (e.g., zinc finger domains, transcription activator-like effector domains) or by guide RNAs (gRNAs).

For specific nucleases described herein, the named protein includes any of the protein's naturally occurring forms, or variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

Zinc Finger Nucleases

In some embodiments, a site-specific nuclease cleavage site is a zinc finger nuclease (ZFN) cleavage site. ZFNs are composed of a zinc-finger DNA-binding domain and a nuclease domain. The DNA-binding domains of individual ZFNs generally contain 3-6 individual zinc finger repeats that recognize 9-18 nucleotides. For example, if the zinc finger domain perfectly recognizes a 3 base pair sequence, then a 3 zinc finger array can be generated to recognize a 9 base pair target DNA sequence. Because individual zinc fingers recognize relatively short (e.g., 3 base pairs) target DNA sequences, ZFNs with 4, 5, or 6 zinc finger domains are typically used to minimize off-target DNA cutting. Non-limiting examples of zinc finger DNA-binding domains that may be used with methods of the present disclosure include Zif268, Ga14, HIV nucleocapsid protein, MYST family histone acetyltransferases, myelin transcription factor Mytl, and suppressor of tumurigenicity protein 18 (ST18). A ZFN may contain homogeneous DNA binding domains (all from the same source molecule) or a ZFN may contain heterogeneous DNA binding domains (at least one DNA binding domain is from a different source molecule).

Zinc finger DNA-binding domains work in concert with a nuclease domain to form ZFNs that cut target DNA. The nuclease cuts the DNA in a non-sequence specific manner after being recruited to the target DNA by the zinc fingers DNA-binding domains. The most widely-used ZFN is the type II restriction enzyme FokI, which forms a heterodimer before producing a double-stranded break in the DNA. Thus, two ZFN proteins bind to opposite strands of DNA to create the FokI heterodimer and form a double-stranded break, reducing off-target DNA cleavage events (Kim, et al., Proc Natl Acad Sci USA, 1996, 93(3): 1156-1160). Additionally, ZFNs may be nickases that only cleave one strand of the double-stranded DNA. By cleaving only one strand, the DNA is more likely to be repaired by error-free HR as opposed to error-prone NHEJ (Ramirez, et al., Nucleic Acids Research, 40(7): 5560-5568). Non-limiting examples of nucleases that may be used as provided herein include FokI and DNaseI.

It should be understood that a ZFN may be expressed as a fusion protein, with the DNA-binding domain and the nuclease domain expressed in the same polypeptide. This fusion may include a linker of amino acids (e.g., 1, 2, 3, 4, 5, 6, or more) between the DNA-binding domain and the nuclease domain.

Transcription Activator-Like Effector Nucleases

Methods described herein, in some embodiments, include the use of transcription activator-like effector nucleases (TALENs) to genetically modify genomic DNA. A TALEN is an endonuclease that can be programmed to cut specific sequences of DNA. TALENs are composed of transcription activator-like effector (TALE) DNA-binding domains, which recognize single target nucleotides in the DNA, and transcription activator-like effector nucleases (TALENs) which cut the DNA at or near a target nucleotide.

Transcription activator-like effectors (TALEs) found in bacteria are modular DNA binding domains that include central repeat domains made up of repetitive sequences of residues (Boch J. et al. Annual Review of Phytopathology 2010; 48: 419-36; Boch J Biotechnology 2011; 29(2): 135-136). The central repeat domains, in some embodiments, contain between 1.5 and 33.5 repeat regions, and each repeat region may be made of 34 amino acids; amino acids 12 and 13 of the repeat region, in some embodiments, determines the nucleotide specificity of the TALE and are known as the repeat variable diresidue (RVD) (Moscou M J et al. Science 2009; 326 (5959): 1501; Juillerat A et al. Scientific Reports 2015; 5: 8150). Unlike ZF DNA sensors, TALE-based sequence detectors can recognize single nucleotides. In some embodiments, combining multiple repeat regions produces sequence-specific synthetic TALEs (Cermak T et al. Nucleic Acids Research 2011; 39 (12): e82). Non-limiting examples of TALEs that may be utilized in the present disclosure include IL2RG, AvrBs, dHax3, and thXoI.

A transcription activator-like effector nuclease (TALEN) cleaves the DNA non-specifically after being recruited to a target sequence by the TALE. This non-specific cleavage can lead to off-target DNA cleavage events. The most widely-used TALEN is the type II restriction enzyme FokI, which forms a heterodimer to produce a double-stranded break in DNA. Thus, two TALEN proteins must bind to opposite strands of DNA to create the FokI heterodimer and form a double-stranded break, reducing off-target DNA cleavage events (Christian M et al. Genetics 2010; 186: 757-761). Additionally, TALEN nucleases may be nickases, which cut only a single-strand of the DNA, thus promoting repair of the break by HR (Gabsalilow L. et al. Nucleic Acids Res. 41, e83). Non-limiting examples of TALENs that may be utilized in the present disclosure include FokI, RNAseH, and MutH.

It should be understood that the TALEN may be expressed as a fusion protein, with the DNA-binding domain and the nuclease domain expressed in the same polypeptide. This fusion may include a linker of amino acids (e.g., 1, 2, 3, 4, 5, 6, or more) between the DNA-binding domain and the nuclease domain.

CRISPR/Cas Nucleases

In some embodiments, a programmable nuclease is a catalytically-active RNA-guided nuclease, such as a Clustered Regularly Interspace Palindromic Repeats (CRISPR/Cas) nuclease. CRISPR/Cas nucleases exist in a variety of bacterial species, where they recognize and cut specific DNA sequences. The CRISPR/Cas nucleases are grouped into two classes. Class 1 systems use a complex of multiple CRISPR/Cas proteins to bind and degrade nucleic acids, whereas Class 2 systems use a large, single protein for the same purpose. A CRISPR/Cas nuclease as used herein may be selected from Cas9, Cas10, Cas3, Cas4, C2c1, C2c3, Cas13a, Cas13b, Cas13c, and Cas14 (e.g., Harrington, L. B. et al., Science, 2018).

CRISPR/Cas nucleases from different bacterial species have different properties (e.g., specificity, activity, binding affinity). In some embodiments, orthogonal catalytically-active RNA-guided nuclease species are used. Orthogonal species are distinct species (e.g., two or more bacterial species). For example, a first catalytically-active Cas9 nuclease as used herein may be a Neisseria meningitidis Cas9 and a second catalytically-active Cas9 nuclease as used herein may be a Streptococcus thermophilus Cas9.

Non-limiting examples of bacterial CRISPR/Cas9 nucleases for use herein include Streptococcus thermophilus Cas9, Streptococcus thermophilus Cas10, Streptooccus thermophilus Cas3, Staphylococcus aureus Cas9, Staphylococcus aureus Cas10, Staphylococcus aureus Cas3, Neisseria meningitidis Cas9, Neisseria meningitidis Cas10, Neisseria meningitidis Cas3, Streptococcus pyogenes Cas9, Streptococcus pyogenes Cas10, and Streptococcus pyogenes Cas3.

A “Cas9 nuclease” herein includes any of the recombinant or naturally-occurring forms of the CRISPR-associated protein 9 (Cas9) or variants or homologs thereof that maintain Cas9 enzyme activity (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 nuclease. In some embodiments, a Cas9 nuclease is substantially identical to the protein identified by the UniProt reference number Q99ZW2 or a variant or homolog having substantial identity thereto.

Other RNA-Guided Nucleases

Other RNA-guided nucleases may be used as provided herein. For example, a CRISPR-associated endonuclease from Prevotella and Francisella 1 (Cpf1) may be used. Cpf1 is a bacterial endonuclease similar to Cas9 nuclease in terms of activity. However, Cpf1 is typically used with a short (˜42 nucleotide) gRNA, while Cas9 is typically used with a longer (˜100 nucleotide) gRNA. Additionally, Cpf1 cuts the DNA 5′ to the target sequence and leaves blunted ends, while Cas9 leaves sticky ends with DNA overhangs. Cpf1 proteins from Acidaminococcus and Lachnospiraceae bacteria efficiently cut DNA in human cells in vitro. In some embodiments, a RNA-guided nuclease is Acidaminococcus Cpf1 or Lachnospiraceae Cpf1, which require shorter gRNAs than Cas nucleases.

### Guide RNAs

A guide RNA (gRNA) is an RNA that binds to an RNA-guided nuclease, such as Cas9, and binds to a target site. Thus, a gRNA includes a targeting sequence and a nuclease-binding sequence. In some embodiments, a gRNA is complexed with an RNA-guided nuclease (e.g., Cas9), which is referred to as a ribonucleoprotein (RNP) complex. A gRNA is designed to bind to only one target site within a defined region (e.g., to locus with a 1 kb region or within an entire genome). That is, a gRNA is designed to include a targeting sequence that is complementary to only one sequence within a defined region (or within a genome of a cell). Nonetheless, as is known in the art, even though a gRNA is designed to be unique to a particular locus, it may bind “off-target” (an unintended target), in some instances.

Targeting Sequence

A targeting sequence of a gRNA comprises a nucleotide sequence that is complementary to a specific sequence within the target site (or to the complementary strand of a target site). Thus, through the targeting sequence, a gRNA binds to a target sit in a sequence-specific manner via hybridization (i.e., base pairing). A protospacer adjacent motif (PAM) is a short specific sequence following a target sequence within a target site that is recognized for cleavage by Cas nuclease. A PAM, in some embodiments, is 2-6 nucleotides downstream of a target sequence bound by a gRNA and a Cas nuclease cuts 3-4 nucleotides upstream of the PAM (e.g., 5′-NGG-3′, wherein N is any deoxyribonucleotide).

The length of a targeting sequence may vary. In some embodiments, the length of a targeting sequence is 10 to 100 nucleotides. For example, a targeting sequence of a gRNA may have a length of 10 to 90, 10 to 80, 10 to 70, 10 to 60, 10 to 50, 10 to 40, 10 to 30, 10 to 20, 15 to 90, 15 to 80, 15 to 70, 15 to 60, 15 to 50, 15 to 40, 15 to 30, 15 to 20, 20 to 90, 20 to 80, 20 to 70, 20 to 60, 20 to 50, 20 to 40, or 20 to 30 nucleotides.

It should be understood that complementarity includes perfect and imperfect complementarity. Thus, in some embodiments, a targeting sequence of a gRNA is 100% identical to a corresponding target sequence within a target site. In other embodiments, however, a targeting sequence of a gRNA is shares less than 100% identity to a corresponding target sequence within a target site. In some embodiments, a targeting sequence shares at least 75% (e.g., at least 80%, at least 85%, at least 90%, at least 95%, at least 97%, at least 98%, or at least 99%) identity with a target sequence.

Nuclease-Binding Sequence

A nuclease-binding sequence of a gRNA binds to a RNA-guided nuclease (e.g., Cas9). In some embodiments, a nuclease-binding sequence comprises two complementary stretches of nucleotides that hybridize to one another to form a double stranded RNA duplex (a dsRNA duplex). These two complementary stretches of nucleotides may be covalently linked by intervening nucleotides known as linkers or linker nucleotides (e.g., in the case of a single-molecule polynucleotide), and hybridize to form the double stranded RNA duplex (dsRNA duplex, or “Cas9-binding hairpin”) of the programmable nuclease-binding sequence, thus resulting in a stem-loop structure.

The length of a nuclease-binding sequence may vary. In some embodiments, the length of a nuclease-binding sequence is 10 to 100 nucleotides. For example, a nuclease-binding sequence of a gRNA may have a length of 10 to 90, 10 to 80, 10 to 70, 10 to 60, 10 to 50, 10 to 40, 10 to 30, 10 to 20, 15 to 90, 15 to 80, 15 to 70, 15 to 60, 15 to 50, 15 to 40, 15 to 30, 15 to 20, 20 to 90, 20 to 80, 20 to 70, 20 to 60, 20 to 50, 20 to 40, or 20 to 30 nucleotides.

Non-limiting examples of nucleotide sequences that can be included in a nuclease-binding sequence are set forth in SEQ ID NOs: 563-682 of WO 2013/176772 (see, e.g., FIGS. 8 and 9 of WO 2013/176772), incorporated herein by reference.

In some cases, a nuclease-binding sequence comprises a nucleotide sequence that differs by 1, 2, 3, 4, or 5 nucleotides from any one of the nuclease-binding sequences described or contemplated herein.

### Target Nucleic Acids

The methods, compositions, and kits of the present disclosure may be used, in some embodiments, to modify a target site. A target site may include any nucleic acid sequence (e.g., DNA sequence) of interest. In some embodiments, a target site is a genomic target site (located in the genome of a cell). A target site includes a site-specific nuclease cleavage site (e.g., a meganuclease cleavage site and/or a programmable nuclease cleavage site).

A target site, in some embodiments, is located within a gene of interest or a regulatory sequence that controls transcription of a gene of interest (e.g., a transcriptional regulatory sequence). For example, a target site may be located within a gene promoter, enhancer, or silencer.

In some embodiments, a target site comprises a DNA sequence. In other embodiments, a target site comprises a RNA sequence.

In some embodiments, a target site is associated with a disorder (e.g., a disease), such as a disorder characterized by a genetic modification. For example, in some embodiments, a target site is within or associated with an oncogene or a tumor suppressor. In some embodiments, a target site comprises a single nucleotide variant (SNV).

Non-limiting examples of disorders and associated genomic loci that may be targeted using the methods and compositions described herein are provided in Table 1.

TABLE 1

| Disorder | Chromosome or gene |
|---|---|
| 1p36 deletion syndrome | 1p36 |
| 18p deletion syndrome | 18p |
| 21-hydroxylase deficiency | 6p21.3 |
| 47, XXX (triple X syndrome) | X |
| AAA syndrome(achalasia-addisonianism-alacrima | AAAS |
| syndrome) | |
| Aarskog-Scott syndrome | FGD1 |
| ABCD syndrome | EDNRB |
| Aceruloplasminemia | CP (3p26.3) |
| Acheiropodia | LMBR1 |
| Achondrogenesis type II | COL2A1 (12q13.11) |
| achondroplasia | FGFR3 (4p16.3) |
| Acute intermittent porphyria | HMBS |
| adenylosuccinate lyase deficiency | ADSL |
| Adrenoleukodystrophy | ABCD1 (X) |
| ADULT syndrome | TP63 |
| Aicardi-Goutières syndrome | TREX1, RNASEH2A, RNASEH2B, |
| | RNASEH2C, SAMHD1, ADAR, IFIH1 |
| Alagille syndrome | JAG1, NOTCH2 |
| Alexander disease | GFAP |
| alkaptonuria | HGD |
| Alpha 1-antitrypsin deficiency | 14q32 |
| Alport syndrome | 10q26.13 COL4A3, COL4A4, |
| | and COL4A5 |
| Alström syndrome | ALMS1 |
| Alternating hemiplegia of childhood | ATP1A3 |
| Alzheimer's disease | PSEN1, PSEN2, APP, APOEε4 |
| Aminolevulinic acid dehydratase deficiency porphyria | ALAD |
| Amyotrophic lateral sclerosis - Frontotemporal dementia | C9orf72, SOD1, FUS, TARDBP, |
| | CHCHD10, MAPT |
| Angelman syndrome | UBE3A |
| Apert syndrome | FGFR2 |
| Arthrogryposis-renal dysfunction-cholestasis syndrome | VPS33B |
| Ataxia telangiectasia | ATM |
| Axenfeld syndrome | PITX2, FOXO1A, FOXC1, PAX6 |
| Beare-Stevenson cutis gyrata syndrome | 10q26, FGFR2 |
| Beckwith-Wiedemann syndrome | IGF-2, CDKN1C, H19, KCNQ1OT1 |
| biotinidase deficiency | BTD |
| Birt-Hogg-Dubé syndrome | 17 FLCN |
| Björnstad syndrome | BCS1L |
| Bloom syndrome | 15q26.1 |
| Brody myopathy | ATP2A1 |
| Brunner syndrome | MAOA |
| CADASIL syndrome | NOTCH3 |
| Campomelic dysplasia | X 17q24.3-q25.1 |
| Canavan disease | ASPA |
| CARASIL syndrome | HTRA1 |
| Carpenter Syndrome | RAB23 |
| Cerebral dysgenesis-neuropathy-ichthyosis-keratoderma | SNAP29 |
| syndrome(SEDNIK) | |
| Charcot-Marie-Tooth disease | PMP22, MFN2 |
| CHARGE syndrome | CHD7 |
| Chédiak-Higashi syndrome | LYST |
| Cleidocranial dysostosis | RUNX2 |
| Cockayne syndrome | ERCC6, ERCC8 |
| Coffin-Lowry syndrome | X RPS6KA3 |
| Cohen syndrome | COH1 |
| collagenopathy, types II and XI | COL11A1, COL11A2, COL2A1 |
| Congenital insensitivity to pain with anhidrosis(CIPA) | NTRK1 |
| Congenital Muscular Dystrophy | multiple |
| Cornelia de Lange syndrome (CDLS) | HDAC8, SMC1A, NIPBL, SMA3, |
| | RAD21 |
| Cowden syndrome | PTEN |
| CPO deficiency (coproporphyria) | CPOX |
| Cranio-lenticulo-sutural dysplasia | 14q13-q21 |
| Cri du chat | 5p |
| Crohn's disease | 16q12 |
| Crouzon syndrome | FGFR2, FGFR3 |
| Crouzonodermoskeletal syndrome (Crouzon syndrome with | FGFR3 |
| acanthosis nigricans) | |
| Cystic fibrosis | CFTR (7q31.2) |
| Darier's disease | ATP2A2 |
| De Grouchy syndrome | 18q |
| Dent's disease (Genetic hypercalciuria) | Xp11.22 CLCN5, OCRL |
| Denys-Drash syndrome | WT1 |
| Di George's syndrome | 22q11.2 |
| Distal hereditary motor neuropathies, multiple types | HSPB8, HSPB1, HSPB3, GARS, REEP1, |
| | IGHMBP2, SLC5A7, DCTN1, TRPV4, |
| | SIGMAR1 |
| Distal muscular dystrophy | Dysferlin, TIA1, GNE (gene), |
| | MYH7, Titin, MYOT, MATR3, |
| | unknown |
| Down Syndrome | 21 |
| Dravet syndrome | SCN1A, SCN2A |
| Duchenne muscular dystrophy | Dystrophin |
| Edwards Syndrome | 18 |
| Ehlers-Danlos syndrome | COL1A1, COL1A2, COL3A1, COL5A1, |
| | COL5A2, TNXB, ADAMTS2, PLOD1, |
| | B4GALT7, DSE |
| Emery-Dreifuss syndrome | EMD, LMNA, SYNE1, SYNE2, FHL1, |
| | TMEM43 |
| Epidermolysis bullosa | KRT5, KRT14, DSP, PKP1, JUP, PLEC1, |
| | DST, EXPH5, TGM5, LAMA3, LAMB3, |
| | LAMC2, COL17A1, ITGA6, ITGA4, |
| | ITGA3, COL7A1, FERMT1 |
| Erythropoietic protoporphyria | FECH |
| Fabry disease | GLA (Xq22.1) |
| Familial adenomatous polyposis | APC |
| Familial Creutzfeld-Jakob Disease | PRNP |
| Familial dysautonomia | IKBKAP |
| Fanconi anemia (FA) | FANCA, FANCB, FANCC, FANCD1, FANCD2, |
| | FANCE, FANCF, FANCG, FANCI, FANCJ, FANCL, |
| | FANCM, FANCN, FANCP, FANCS, RAD51C, XPF |
| Fatal familial insomnia | PRNP |
| Feingold syndrome | MYCN |
| FG syndrome | MED12 |
| Fragile X syndrome | FMR1 |
| Friedreich's ataxia | FXN |
| Galactosemia | GALT, GALK1, GALE |
| Gaucher disease | GBA (1) |
| Gerstmann-Sträussler-Scheinker syndrome | PRNP |
| Gillespie syndrome | PAX6 |
| Glutaric aciduria, type Iand type 2 | GCDH, ETFA, ETFB, ETFDH |
| GRACILE syndrome | BCS1L |
| Griscelli syndrome | MYO5A, RAB27A, MLPH |
| Hailey-Hailey disease | ATP2C1 (3) |
| Harlequin type ichthyosis | ABCA12 |
| Hemochromatosis, hereditary | HFE, HAMP, HFE2B, TFR2, TF, CP |
| Hemophilia | FVIII |
| Hepatoerythropoietic porphyria | UROD |
| Hereditary coproporphyria | 3q12 |
| Hereditary hemorrhagic telangiectasia (Osler-Weber-Rendu | ENG, ACVRL1, MADH4 |
| syndrome) | |
| Hereditary inclusion body myopathy | GNE, MYHC2A, VCP, HNRPA2B1, |
| | HNRNPA1 |
| Hereditary multiple exostoses | EXT1, EXT2, EXT3 |
| Hereditary neuropathy with liability to pressure | PMP22 |
| palsies (HNPP) | |
| Hermansky-Pudlak syndrome | HPS1, HPS3, HPS4, HPS5, HPS6, HPS7, |
| | AP3B1 |
| Heterotaxy | NODAL, NKX2-5, |
| | ZIC3, CCDC11, CFC1, SESN1 |
| Homocystinuria | CBS (gene) |
| Hunter syndrome | IDS |
| Huntington's disease | HD |
| Hurler syndrome | IDUA |
| Hutchinson-Gilford progeria syndrome | LMNA |
| Hyperlysinemia | AASS |
| Hyperoxaluria, primary | AGXT, GRHPR, DHDPSL |
| Hyperphenylalaninemia | 12q |
| Hypoalphalipoproteinemia(Tangier disease) | ABCA1 |
| Hypochondrogenesis | COL2A1 |
| Hypochondroplasia | FGFR3 (4p16.3) |
| Immunodeficiency-centromeric instability-facial anomalies | 20q11.2 |
| syndrome (ICF syndrome) | |
| Incontinentia pigmenti | IKBKG (Xq28) |
| Ischiopatellar dysplasia | TBX4 |
| Isodicentric 15 | 15q11-14 |
| Jackson-Weiss syndrome | FGFR2 |
| Joubert syndrome | INPP5E, TMEM216, AHI1, NPHP1, CEP290, |
| | TMEM67, RPGRIP1L, ARL13B, CC2D2A, |
| | OFD1, TMEM138, TCTN3, ZNF423, AMRC9 |
| Juvenile primary lateral sclerosis (JPLS) | ALS2 |
| Kniest dysplasia | COL2A1 |
| Kosaki overgrowth syndrome | PDGFRB |
| Krabbe disease | GALC |
| Kufor-Rakeb syndrome | ATP13A2 |
| LCAT deficiency | LCAT |
| Lesch-Nyhan syndrome | HPRT (X) |
| Li-Fraumeni syndrome | TP53 |
| Limb-Girdle Muscular Dystrophy | Multiple |
| Lynch syndrome | MSH2, MLH1, MSH6, PMS2, PMS1, |
| | TGFBR2, MLH3 |
| Malignant hyperthermia | RYR1 (19q13.2) |
| Maple syrup urine disease | BCKDHA, BCKDHB, DBT, DLD |
| Maroteaux-Lamy syndrome | ARSB |
| McCune-Albright syndrome | 20 q13.2-13.3 |
| McLeod syndrome | XK(X) |
| Mediterranean fever, familial | MEFV |
| MEDNIK syndrome | AP1S1 |
| Menkes disease | ATP7A (Xq21.1) |
| Methylmalonic acidemia | MMAA, MMAB, MMACHC, MMADHC, |
| | LMBRD1, MUT |
| Micro syndrome | RAB3GAP (2q21.3) |
| Microcephaly | ASPM(1q31) |
| Morquio syndrome | GALNS, GLB1 |
| Mowat-Wilson syndrome | ZEB2 (2) |
| Muenke syndrome | FGFR3 |
| Multiple endocrine neoplasia type 1(Wermer's syndrome) | MEN1 |
| Multiple endocrine neoplasia type 2 | RET |
| Muscular dystrophy | multiple |
| Myostatin-related muscle hypertrophy | MSTN |
| myotonic dystrophy | DMPK, CNBP |
| Natowicz syndrome | HYAL1 |
| Neurofibromatosis type I | 17q11.2 |
| Niemann-Pick disease | SMPD1, NPA, NPB, NPC1, NPC2 |
| Nonketotic hyperglycinemia | GLDC, AMT, GCSH |
| Nonsyndromic deafness | |
| Noonan syndrome | PTPN11, KRAS, SOS1, RAF1, NRAS, |
| | HRAS, BRAF, SHOC2, MAP2K1, MAP2K2, CBL |
| Norman-Roberts syndrome | RELN |
| Ogden syndrome | X |
| Omenn syndrome | RAG1, RAG2 |
| Osteogenesis imperfecta | COL1A1, COL1A2, IFITM5 |
| Pantothenate kinase-associated neurodegeneration | PANK2 (20p13-p12.3) |
| Patau syndrome (Trisomy 13) | 13 |
| PCC deficiency (propionic acidemia) | PC |
| Pendred syndrome | PDS (7) |
| Peutz-Jeghers syndrome | STK11 |
| Pfeiffer syndrome | FGFR1, FGFR2 |
| Phenylketonuria | PAH |
| Pipecolic acidemia | AASDHPPT |
| Pitt-Hopkins syndrome | TCF4(18) |
| Polycystic kidney disease | PKD1 (16) or PKD2 (4) |
| Porphyria cutanea tarda(PCT) | UROD |
| Prader-Willi syndrome | 15 |
| Primary ciliary dyskinesia(PCD) | DNAI1, DNAH5, TXNDC3, DNAH11, |
| | DNAI2, KTU, RSPH4A, RSPH9, LRRC |
| | 50 |
| Protein C deficiency | PROC |
| Protein S deficiency | PROS1 |
| Pseudoxanthoma elasticum | ABCC6 |
| Retinitis pigmentosa | RP1, RP2, RPGR, PRPH2, IMPDH1, |
| | PRPF31, CRB1, PRPF8, TULP1, CA4, |
| | HPRPF3, ABCA4, EYS, CERKL, |
| | FSCN2, TOPORS, SNRNP200, PRCD, |
| | NR2E3, MERTK, USH2A, PROM1, |
| | KLHL7, CNGB1, TTC8, ARL6, |
| | DHDDS, BEST1, LRAT, SPARA7, |
| | CRX |
| Rett syndrome | MECP2 |
| Roberts syndrome | ESCO2 |
| Rubinstein-Taybi syndrome (RSTS) | CREBBP |
| Sandhoff disease | HEXB |
| Sanfilippo syndrome | SGSH, NAGLU, HGSNAT, GNS |
| Schwartz-Jampel syndrome | HSPG2 |
| Shprintzen-Goldberg syndrome | FBN1 |
| Sickle cell anemia | 11p15 |
| Siderius X-linked mental retardation syndrome | PHF8 |
| Sideroblastic anemia | ABCB7, SLC25A38, GLRX5 |
| Sjogren-Larsson syndrome | ALDH3A2 |
| Sly syndrome | GUSB |
| Smith-Lemli-Opitz syndrome | DHCR7 |
| Smith-Magenis syndrome | 17p11.2 |
| Snyder-Robinson syndrome | Xp21.3-p22.12 |
| Spinal muscular atrophy | 5q |
| Spinocerebellar ataxia(types 1-29) | ATXN1, ATXN2, ATXN3, PLEKHG4, |
| | SPTBN2, CACNA1A, ATXN7, ATXN8OS, |
| | ATXN10, TTBK2, PPP2R2B, KCNC3, |
| | PRKCG, ITPR1, TBP, KCND3, FGF14 |
| Spondyloepiphyseal dysplasia congenita(SED) | COL2A1 |
| SSB syndrome (SADDAN) | FGFR3 |
| Stargardt disease(macular degeneration) | ABCA4, CNGB3, ELOVL4, PROM1 |
| Stickler syndrome(multiple forms) | COL11A1, COL11A2, COL2A1, COL9A1 |
| Strudwick syndrome (spondyloepimetaphyseal dysplasia, | COL2A1 |
| Strudwick type) | |
| Tay-Sachs disease | HEXA (15) |
| Tetrahydrobiopterin deficiency | GCH1, PCBD1, PTS, QDPR, MTHFR, |
| | DHFR |
| Thanatophoric dysplasia | FGFR3 |
| Treacher Collins syndrome | 5q32-q33.1 (TCOF1, POLR1C, |
| | or POLR1D) |
| Tuberous sclerosis complex (TSC) | TSC1,TSC2 |
| Turner syndrome | X |
| Usher syndrome | MYO7A, USH1C, CDH23, PCDH15, |
| | USH1G, USH2A, GPR98, DFNB31, |
| | CLRN1 |
| Variegate porphyria | PPOX |
| von Hippel-Lindau disease | VHL |
| Waardenburg syndrome | PAX3, MITF, WS2B, WS2C, SNAI2, |
| | EDNRB, EDN3, SOX10 |
| Weissenbacher-Zweymüller syndrome | COL11A2 |
| Williams syndrome | 7q11.23 |
| Wilson disease | ATP7B |
| Wolf-Hirschhorn syndrome | 4p16.3 |
| Woodhouse-Sakati syndrome | C2ORF37 (2q22.3-q35) |
| X-linked intellectual disability and macroorchidism (fragile | X |
| X syndrome) | |
| X-linked severe combined immunodeficiency (X-SCID) | X |
| X-linked sideroblastic anemia (XLSA) | ALAS2 (X) |
| X-linked spinal-bulbar muscle atrophy (spinal and bulbar | X |
| muscular atrophy) | |
| Xeroderma pigmentosum | 15 ERCC4 |
| Xp11.2 duplicationsyndrome | Xp11.2 |
| XXXX syndrome (48, XXXX) | X |
| XXXXX syndrome (49, XXXXX) | X |
| XYY syndrome (47, XYY) | X |
| Zellweger syndrome | PEX1, PEX2, PEX3, PEX5, PEX6, PEX10, |
| | PEX12, PEX13, PEX14, PEX16, PEX19, |
| | PEX26 |

### Cells

Aspects of the present disclosure provide cells (e.g., host cells) comprising a donor nucleic acid that includes a chemical modification within an internal region that comprises a site-specific nuclease cleavage site. A cell, in some embodiments, expresses the site-specific nuclease (e.g. genomically encode the site-specific nuclease) that can cleave the target site. In other embodiments, a nucleic acid encoding the site-specific nuclease is delivered to a cell.

Methods of delivery are known, any of which may be used as provided herein. Non-limiting examples of methods of delivery of a protein or nucleic acid to a cell include viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, and nanoparticle-mediated nucleic acid delivery (see, e.g., Panyam et al., Adv. Drug Deliv. Rev., pii: 50169-409X(12)00283-9.doi:10.1016/j.addr.2012.09.023). In some embodiments, a donor nucleic acid, site-specific nuclease or nucleic acid encoding a site-specific nuclease, and/or a gRNA or a nucleic acid encoding a gRNA is/are delivered to a cell via electroporation. In some embodiments, a donor nucleic acid, site-specific nuclease or nucleic acid encoding a site-specific nuclease, and/or a gRNA or a nucleic acid encoding a gRNA is/are delivered to a cell via transfection, e.g., using a cell transfection reagent.

A nucleic acid that is delivered to a cell may be present on a vector, for example, a viral vector (e.g., adenoviral vector or adeno-associated viral vector) or a plasmid vector. In some embodiments, a viral vector is derived from Adenoviridae, Parvoviridae, Togaviridae, Herpesviridae, Retroviridae, or Poxviridae. Other vectors may be used. An expression vector (e.g., a recombinant expression vector) typically includes a promoter, such as an inducible promoter. In some embodiments, an expression vector comprises a promoter, a translation initiation sequence such as a ribosomal binding site and start codon, a termination codon, and/or a transcription termination sequence.

A cell may be a eukaryotic cell or a prokaryotic cell. In some embodiments, a cell is a mammalian cell. In some embodiments, a cell is a primate cell. In some embodiments, a cell is a human cell. In some embodiments, a mammalian cell is a rodent cells, such as a mouse cell or a rat cell. In some embodiments, a cell is bacterial cell (e.g., Escherichia coli cell). In some embodiments, a cell is yeast cell (e.g., Saccharomyces cerevisiae cell).

A cell, in some embodiments, is a pluripotent cell. A stem cell is an example of a pluripotent cell. Non-limiting examples of pluripotent stem cells include embryonic stem cells and adult stem cells. In some embodiments, a pluripotent stem cell is an induced pluripotent stem cell (iPSC) (e.g., adult cell that has been genetically reprogrammed to an embryonic stem cell-like state). In some embodiments an iPSC cell is a human iPSC cell.

A cell, in some embodiments, is an epithelial cell, a nerve cell, a muscle cell, or a connective tissue cell.

A cell, in some embodiments, is selected from the group consisting of stem cells, bone cells, blood cells, muscle cells, fat cells, skin cells, nerve cells, endothelial cells, sex cells, pancreatic cells, and cancer cells. In some embodiments, a cell is a cancer cell.

In some embodiments, a cell is a zygote. A zygote is a diploid cell resulting from the fusion of two haploid gametes (a fertilized ovum). In some embodiments, a zygote is selected a 1-cell stage zygote. In some embodiments, a zygote is selected a 2-cell stage zygote. In some embodiments, a zygote is selected a 4-cell stage zygote. In some embodiments, a zygote is selected an 8-cell stage zygote. The present disclosure also contemplates the delivery of a donor nucleic acid (and any of the other nucleic acids and/or nucleases provided herein) to a later stage zygote, for example, to a morula (e.g., ˜72 hours of development) or to a blastocyte (e.g., ˜4-5 days of development). In some embodiments, a zygote is a human zygote. In some embodiments, a zygote is a primate zygote. In some embodiments, a zygote is a rodent zygote (e.g., a mouse zygote). In some embodiments, a donor nucleic acid (and any of the other nucleic acids and/or nucleases provided herein) is delivered to an embryo.

In some embodiments, a cell is maintained under conditions that result in cleavage of the target site. In some embodiments, a cell is maintained under conditions that result in the target site comprising the chemical modification (and is thus resistant to site-specific nuclease cleavage).

Such conditions may include a cold shock, which refers to culturing a cell at a certain temperature, then culturing the cell at a lower temperature. For example, a cell may be cultured at 37° C., and the following delivery of a donor nucleic acid (and any other components), the cell may be further cultured at 32° C., and then again at 37° C. In some embodiments, the cold shock period is 12 hours, 1 day, 1.5 days, 2 days, 2.5 days, 3 days, 3.5 days, or 4 days. In some embodiment, the cold shock period is two days. See, e.g., Skarnes W C et al. Methods, 2019; vol. 164-165: pp. 18-28.

In some embodiments, conditions further include a small molecule enhancer of homology directed repair (HDR). Small molecule enhancers of HDR are known in the art and include, but are not limited to, H7904, A2169, B7651, SML1362, SML1546, M1404, R9782, SCR7, MLN924, NSC15520, AZD7762, VE822, and ALT-R® HDR enhancer (IDT). In some embodiments, a small molecule enhancer of HDR is ALT-R® HDR. Such conditions are described in, Skarnes W C et al. Methods, 2019; vol. 164-165: pp. 18-28.

### Kits

The present disclosure also provides kits, for example, for performing any one of the methods described herein. A kit, in some embodiments, comprises a donor nucleic acid of the present disclosure. In some embodiments, a kit further comprises a site-specific nuclease (e.g., a meganuclease and/or a programmable nuclease) or a nucleic acid encoding a site-specific nuclease. In some embodiments, a kit further comprises a programmable nuclease selected from the group consisting of Cas9 nucleases, ZFNs, and TALENs. In some embodiments, a kit further comprises a gRNA or a nucleic acid encoding a gRNA.

A donor nucleic acid and any one or more of the nucleic acids of a kit may be encoded on the same vector or on different vectors (e.g., expression vectors).

In some embodiments, a kit also comprises at least one reagent that facilitates the delivery of a nucleic acid or protein into a cell (e.g., a cell transformation, transfection, infection, or electroporation reagent).

Additional kit reagents may be selected from the group consisting of: a buffer; a wash buffer; a control reagent; a control expression vector or RNA nucleic acid; and a reagent for in vitro production of site-specific nuclease (e.g., Cas9).

Components of a subject kit can be in separate containers or can be combined in a single container.

In addition to above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example 1. Cleavage-Resistant Templates for High-Efficiency Editing and Control of Zygosity

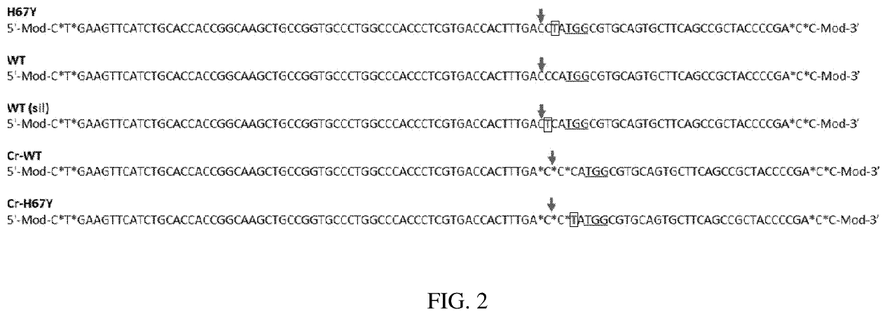

End-modified single-stranded oligonucleotides (ssODNs) were generated for oligo mixing experiments. The ssODNs included the wildtype (“WY”) blue fluorescent protein (BFP) sequence, a single nucleotide variant converting BFP to green fluorescent protein (GFP) (“H67Y”) sequence, a wildtype BFP sequence containing a silent coding variant (“WT (sil)”), and cleavage-resistant forms of the H67Y variant (“Cr—H67Y”) and the wildtype (“Cr—WY”) sequences. The cleavage-resistant forms each have three phosphorothioate (PS) linkages at the predicted Cas9 cut site, upstream of the PAM sequence. The sequences are illustrated in FIG. 2.

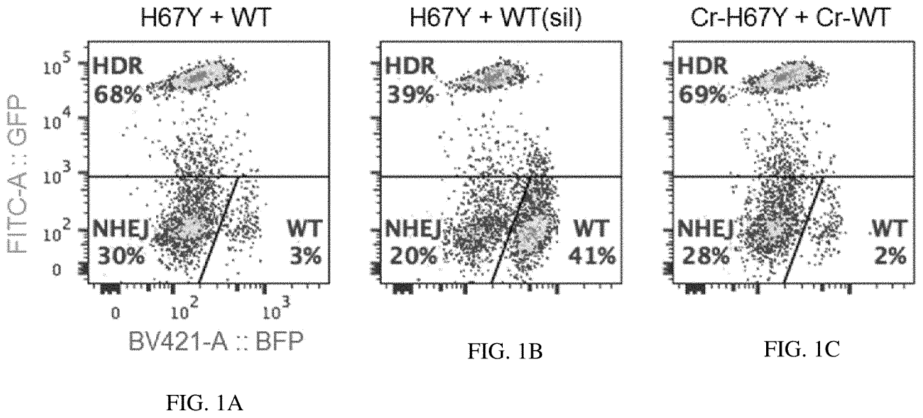

Human iPS cells were nucleotransfected with high-fidelity Cas9 RNP-targeting blue fluorescent protein (BFP) sequences and the ssODNs described above. In the oligo mixing experiments, very few wildtype alleles were produced by homology directed repair (HDR), suggesting that the wildtype sequence is highly susceptible to re-cleavage by Cas9 nucleases (FIG. 1A). However, the inclusion of a “silent” mutation (e.g., a synonymous change in coding sequence) in the wildtype donor nucleic acid is sufficient to suppress re-cleavage and recover equal numbers of mutant and “wildtype” alleles (FIG. 1B). The wildtype alleles were not retained (FIG. 1C), possibly due to repeated rounds of HDR and re-cleavage, favoring the retention of the SNV (GFP-positive) allele over the wildtype allele.

The same ssODNs (presented in FIG. 2) were further examined in a BFP-to-GFP assay. As shown in FIG. 3, the presence of PS linkages does not interfere with the conversion of BFP to GFP (a SNV), indicating that the templates are efficiently incorporated into the genome and do not interfere with the expression of the target gene. In fact, the presence of PS linkages increased the fraction of wildtype (BFP-positive) alleles, from 13% to 22%, illustrating that it is possible to inhibit re-cleavage of the edited allele with a chemically-modified donor nucleic acid described herein.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A donor nucleic acid comprising a chemical modification within an internal region that comprises a site-specific nuclease cleavage site.

2. The donor nucleic acid of claim 1, wherein the chemical modification is with 1 to 10 nucleotides of the site-specific nuclease cleavage site.

3. The donor nucleic acid of claim 2, wherein the chemical modification is with 1 to 5 nucleotides of the site-specific nuclease cleavage site.

4. The donor nucleic acid of claim 3, wherein the chemical modification is within the site-specific nuclease cleavage site.

5. The donor nucleic acid of any one of the preceding claims, wherein the chemical modification is a phosphorothioate (PS) linkage.

6. The donor nucleic acid of any one of the preceding claims, wherein the internal region of the donor nucleic acid comprises 1 to 5 chemical modifications.

7. The donor nucleic acid of any one of the preceding claims, wherein the donor nucleic acid is single stranded.

8. The donor nucleic acid of any one of the preceding claims, wherein the donor nucleic acid comprises a genetic modification relative to a target site.

9. The donor nucleic acid of claim 8, wherein the genetic modification is selected from the group consisting of insertions, deletions, substitutions, and combinations thereof.

10. The donor nucleic acid of claim 8 or 9, wherein the genetic modification is a single nucleotide variant (SNV).

11. The donor nucleic acid of any one of the preceding claims, wherein the site-specific nuclease cleavage site is selected from the group consisting of programmable nuclease cleavage sites and meganuclease cleavage sites.

12. The donor nucleic acid of claim 11, wherein the site-specific nuclease cleavage site is a programmable nuclease cleavage site.

13. The donor nucleic acid of claim 12, wherein the programmable nuclease cleavage site is selected from the group consisting of Cas9 nuclease cleavage sites, zinc finger nuclease (ZFN) cleavage sites, and transcription activator-like effector nuclease (TALEN) cleavage sites.

14. The donor nucleic acid of claim 13, wherein the programmable site-specific nuclease cleavage site is a Cas9 nuclease cleavage site.

15. The donor nucleic acid of any one of the preceding claims, wherein the donor nucleic acid comprises a chemical modification within an end region of the donor nucleic acid.

16. The donor nucleic acid of claim 15, wherein the chemical modification is a PS linkage and/or a 2′O-methyl analog.

17. A kit comprising the donor nucleic acid of any one of the preceding claims.

18. The kit of claim 17 further comprising a site-specific nuclease or a nucleic acid encoding a site-specific nuclease.

19. The kit of claim 18, wherein the site-specific nuclease is selected from the group consisting of programmable nucleases and meganucleases.

20. The kit of claim 19, wherein the site-specific nuclease is a programmable nuclease.

21. The kit of claim 20, wherein the programmable nuclease is selected from the group consisting of Cas9 nucleases, ZFNs, and TALENs.

22. The kit of claim 21, wherein the programmable nuclease is a Cas9 nuclease.

23. The kit of claim 2 further comprising a guide RNA (gRNA) or a nucleic acid encoding a gRNA.

24. A method comprising delivering to a cell that comprises a target site the donor nucleic acid of any one of the preceding claims, wherein the internal region of the donor nucleic acid comprises a sequence homologous to or partially homologous to the target site.

25. A method comprising delivering to a cell that comprises a target site within a target allele a mixture of donor nucleic acids of any one of the preceding claims.

26. The method of claim 25, wherein a subset of donor nucleic acids of the mixture comprise a wild-type allele and a subset of donor nucleic acids of the mixture comprise a single nucleotide variant allele, relative to the target allele; or wherein a subset of donor nucleic acids of the mixture comprise a first single nucleotide variant allele and a subset of donor nucleic acids of the mixture comprise a second single nucleotide variant allele, relative to the target allele.

27. The method of any one of the preceding claims, wherein the cell further comprises a site-specific nuclease that can cleave the target site.

28. The method of any one of the preceding claims, further comprising delivering to the cell a site-specific nuclease or a nucleic acid encoding a site-specific nuclease that can cleave the target site.

29. The method of any one of the preceding claims, further comprising delivering to the cell a guide RNA (gRNA) that can bind to the target site or a nucleic acid encoding a gRNA that can bind to the target site.

30. The method of any one of the preceding claims, further comprising maintaining the cell under conditions that result in cleavage of the target site.

31. The method of claim 30, further comprising maintaining the cell under conditions that result in the target site comprising the chemical modification.

32. The method of claim 30 or 31, wherein the conditions include a cold shock.

33. The method of any one of claims 30-32, wherein the conditions include presence of a small molecule enhancer of homology directed repair (HDR).

34. A cell comprising the donor nucleic acid of any one of the preceding claims and a target site.

35. The cell of claim 34, further comprising a site-specific nuclease or a nucleic acid encoding a site-specific nuclease that can cleave the target site.

36. The cell of claim 34 or 35, further comprising a guide RNA (gRNA) or a nucleic acid encoding a gRNA that can bind to the target site.

37. The cell of any one of the preceding claims, wherein the cell is a human cell or a rodent cell.

38. The cell of any one of the preceding claims, wherein the cell is a stem cell.

39. The cell of claim 38, wherein the stem cell is a pluripotent stem cell.

40. The cell of claim 39, wherein the pluripotent stem cell is an induced pluripotent stem cell (iPSC).

41. A zygote comprising the donor nucleic acid of any one of the preceding claims and a target site.

42. The zygote of claim 41, further comprising a site-specific nuclease or a nucleic acid encoding a site-specific nuclease that can cleave the target site.

43. The zygote of claim 41 or 42, further comprising a guide RNA (gRNA) or a nucleic acid encoding a gRNA that can bind to the target site.

44. The zygote of any one of the preceding claims, wherein the zygote is a human zygote or a rodent zygote.

45. The zygote of any one of the preceding claims, wherein the zygote is selected from 1-cell stage zygotes, 2-cell stage zygotes, 4-cell stage zygotes, and 8-cell stage zygotes.
